(19) United States
(12) Patent Application Publication
(10) Pub. No.: US 2011/0151503 A1
(43) Pub. Date: Jun. 23, 2011

(54) DEVICE AND METHOD FOR BACTERIOLOGICAL TESTING ON PLASMA

(75) Inventor: Paolo Galiano, Padova (IT)

(73) Assignee: Alifax Holding Spa, Polverara (IT)

(21) Appl. No.: 13/060,217

(22) PCT Filed: Aug. 19, 2009
(86) PCT No.: PCT/IB2009/006588

§ 371 Date: Feb. 22, 2011

(30) Foreign Application Priority Data
Aug. 22, 2008 (IT) UD2008A000190

(51) Int. Cl. C12Q 1/06; C12Q 1/04; C12M 1/34

(52) U.S. Cl. 435/39; 435/34; 435/288.7

(57) ABSTRACT

Apparatus for making a bacteriological test on plasma, comprising a sedimentation unit for a blood sample contained in a first container to separate the corpuscular part of the sample, which sediments on the bottom of the first container, from the liquid part or plasma, pick-up and inoculum means to pick up a portion of the surnatant, and to inoculate the portion in a culture ground inside a second container allowing a bacterial growth, optical measurement means, to effect measurements of the culture ground in order to determine the presence of bacteria and microorganisms, and processing means comprising a data bank, to collect measurement data, to construct a curve that represents the intensity of the radiation diverted by the culture ground in the measurement with respect to time, whose parameters are compared with reference values in order to determine typical analysis parameters, said values being characteristic for each bacterial species. Disclosed is further a corresponding method.

### FIELD OF THE INVENTION

The present invention concerns a new procedure for a diagnostic test on a blood sample, which consists of growing possible microorganisms present in the blood and in determining their bacterial load.

### BACKGROUND OF THE INVENTION

It is known that diagnostic analysis on blood, serum and/or plasma can have different objectives and applications. On the other hand, microbiological analysis normally identifies the presence of bacteria or other pathogen microorganisms by means of cultural techniques, since they are potentially responsible for infections in different parts of the human body, and by means of subsequent tests identifies the type (identification test) and determines the sensitivity in vitro to antibiotics (anti-biogram test).

For this last type of test, in the current state of the art, different cultural techniques for the detection of the presence/absence of the number and the types of pathogen organisms are known.

The classical technique (considered historically as a reference) consists in the distribution of known volumes of samples of whole blood, possible diluted, on different solid culture grounds suitable for the proliferation of possible bacteria colonies present in the whole blood, known as Petri dishes. Such cultures are done in conditions of aerobiosis and anaerobiosis.

The semi-quantitative evaluation of the bacteria present in the culture after a certain period of time allows to have approximate indications on the initial concentration of the bacteria, with reference to the volume of the sample used and to the dilution factor possibly used.

However, the time needed for the execution of this test requires an incubation of the Petri dishes extended for some days (normally 5-7 days) with daily readings and checks. Only at the end of this time can the analysis be considered ended and a possible positive result be excluded.

Furthermore, the calculation is mostly based on visible evaluations of the final bacterial distribution and on considerations of a statistical type by the operator, thus not guaranteeing the absolute precision of the method.

Moreover, the known method entails a considerable amount of work both for sowing the various dishes and also for reading them, which has to be done daily above all considering that a considerable percentage of the cultures turn out to be negative.

Later on, with the purpose of simplifying these analytical methods, new partially automated systems were created which detect the presence of microorganisms by means of particular chemical reactions inside the sample, for example by measuring the formation of carbon dioxide or other chemical substances which indicate the presence of bacteria.

These methods provide information on the presence/absence of bacteria and an automated reading of the test, thus allowing to select the positive samples from the negative ones and reducing the work of the operator.

Using these systems it is in any case necessary, for the positive samples, to carry out the cultural procedure on dishes as previously described (subject to very long times) in order to evaluate the type of bacteria detected by the system and to use the isolation of potential pathogens for the execution of the subsequent tests (identification and anti-biogram). Moreover these known methods can be disturbed by non-specific reactions or other variables which affect the reliability of the results obtained.

Furthermore, often, one single sample may not show an intermittent bacteremia and makes it difficult to interpret the clinical significance of the isolation of some microorganisms. For this reason, according to international guide lines, it is necessary to carry out sequential sampling on the same patient.

Purpose of the present invention is to perfect a method for testing haemocultures using the plasma of the sample examined, applying the light scattering technology relating to aerobiosis cultures, able to give precise and reliable results with a considerable saving of time and equipment compared to known methods.

Another purpose of the invention is to perfect an automated method which requires an extremely reduced manual work, thanks to the automation of the reading phases, data processing, display of the results etc.

A further purpose of the present invention is to perfect a method with very high sensitivity in establishing in a short time the presence/absence of bacterial loads in the plasma sample.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

The method according to the present invention provides to use plasma of the blood sample taken from the patient

First of all the blood sample is typically put in contact with an anticoagulant and optionally then lysed to break up the red corpuscles so as to free the potential bacteria inside the red corpuscles.

The blood sample is sedimented to obtain plasma. The plasma is taken and inoculated into a culture medium in a liquid form (eugonic broth) and maintained in continuous agitation so as to facilitate the growth of the bacteria present.

The bacterial growth is measured with technology based on light scattering, with automatic signaling of the McFarland turbidity 0.5, useful, as will be seen hereafter, to be able to do the anti-biogram even without identifying the species. It is possible to carry out the antibiotic functionality tests on the liquid state eugonic broth having a positive result to measurement using the light scattering technique, when McFarland turbidity 0.5 is reached, without waiting for the isolation of the bacteria possibly present, applying the direct anti-biogram to the antibiotics administered by the clinician. It is known that the clinician administers the antibiotic even without bacteriological indications with the purpose of saving the life of the patient under examination.

The mathematical algorithm applied to the detection of the growth curve of the bacteria allows to quantify the bacterial load and to hypothesize the identification of bacterial types based on comparison with growth curves obtained from a data bank, growth curves detected by the same instrument comparing ATCC bacterial strains (standard control bacterial strains deposited at a public data bank).

The method according to the invention uses, as stated, the technique based on light-scattering due to the presence of corpuscular elements (bacteria, fungi) inside a liquid solution.

The Applicant has found that the light-scattering technology is particularly sensitive and therefore suitable to carry out measurements of the quantity of light diffused due to bodies in suspension, such as bacteria and other microorganisms, even when the concentration of the solution is extremely limited.

To achieve this technology and eliminate the interference of the measurement due to the presence of red corpuscles in the culture broth and/or to the high concentration of hemoglobin, the Applicant has focused attention on the search for bacteria and microorganisms, aerobes, microaerophiles or capnophiles present in the plasma sample and, according to a variant, also the presence of bacteria inside the red corpuscles, causing the lysis of the red corpuscles themselves.

The method according to the present invention is advantageously applied to a preliminary investigation to the subsequent execution of an anti-biogram test.

In more detail, the method according to the present invention comprises a first step where a blood sample taken from a patient is dispensed in a first container.

Advantageously, in this step an anticoagulant is added.

As a variant, a lysis operation of the sample contained in the first container is carried out.

The method also comprises the following steps:
a second step in which the sedimentation of the blood sample is determined, in the case of the variant which provides lysis, the sedimentation of the erythrocytes lysed present in the sample, so as to separate the corpuscular part, which sediments on the bottom of the first container, from the liquid part or plasma;
a third step in which a determinate portion of the surnatant is taken, consisting of the liquid part or plasma thus obtained. Typically the greater part of the bacteria and microorganisms associated with the most widespread and common pathologies are contained in the plasma;
a fourth step in which the portion of the liquid part or plasma obtained in a culture ground is inoculated inside a second container suitable to allow a bacterial culture and an instrument reading by means of an optical measurement machine. In particular, it can advantageously be a liquid culture ground, such as a eugonic broth, inside a glass bottle for example, suitable to allow bacterial culture and an instrument reading. The broth is an aqueous solution of grounds able to promote the growth and the proliferation of the microorganisms possibly present in the sample;
a fifth step in which bacterial growth is allowed in the culture ground contained in the second container. In particular, the bottle, already inoculated and housed in the instrument is subjected to thermostating and continuous mixing at 37° C. to promote the possible growth of microorganisms present in the plasma sample;
a sixth step in which, by means of the optical measurement machine, an optical measurement is made on the culture ground contained in the second container, in order to determine the presence of bacteria and microorganisms in the plasma sample. In particular, kinetic optical measurements with fixed timing are made on the second container, based on the light scattering technique, to determine the presence of possible bacterial growth, and subsequently by analyzing the signals the instrument shows the curves of bacterial growth.

Preferably, the optical measurement of the sixth step takes place simultaneously with the fifth step, so as to measure the bacterial growth directly.

By plasma we mean the constituent liquid of the blood subjected to treatment with anticoagulant, in which it is usually present to a percentage of about 55% of the total mass. It is an aqueous solution, yellow in color and of a colloidal nature, containing protein, glucides, lipids and salts.

Advantageously, the optical measurement which is carried out is of the nephelometric type based on light scattering technology. The optical measurement provides a quantitative measurement of the bacterial count, based on an exponential development as a function of time, assuming a development such as C_{B}=Ae^{K}^{ₙ}^{(t-t}^{₀₎}+C.

The optical measurement is very advantageous in that it allows to considerably shorten the analysis times, compared with the known analysis procedures which are based on chemical indicators and subsequent culture in Petri dishes.

Moreover, the optical measurement of the sixth step is able to signal that the McFarland level of turbidity 0.5 has been reached.

In fact, the method is able to signal, by means of monitor display or a sound signal, the possible turbidity corresponding to the growth signal of the bacteria present in the plasma sample under examination, when the McFarland turbidity value of 0.5 has been reached. To this purpose, there is a standard turbidity control latex in the appropriate instrument to be able to signal the McFarland turbidity 0.5 and subsequently carry out the suitable anti-biogram.

It is thus possible to carry out the direct execution of the anti-biogram, using the same eugonic broth as an inoculum ready with the same level of McFarland turbidity 0.5 required, without waiting for the identification of the bacteria.

This is advantageous in that, in the known technique, to carry out the anti-biogram, international guide lines usually require an isolation of the colonies on the Petri dishes and subsequent preparation of a bacterial suspension with a McFarland turbidity level 0.5, obtained by means of diluting the colonies. Therefore in the state of the art one usually starts from a bacterial concentrate which is diluted. Instead, with the present invention, the progressive measuring of the bacterial growth is made, until the McFarland turbidity value 0.5 is reached automatically, allowing a saving in both time and money.

In this way, the steps thus so far described end with the availability of positive samples with a turbidity suitable to begin the clinical anti-biogram, that is, the functionality tests on the antibiotics directly from the growth broth.

This advantage allows to supply the functional result of the first antibiotic tested (resistant or sensitive) to the clinician in order to correctly treat the patient for the antibiotic administered if the result turns out sensitive, or to change the antibiotic if the result shows that it is resistant.

The invention therefore allows to carry out of an anti-biogram of the clinical type, that is, an anti-biogram made directly on the growth broth inoculated with the plasma sample being tested which showed positive to bacterial growth.

Moreover the attainment of the 0.5 McFarland value according to the present invention is much more precise compared with the method where the concentrated sample is diluted, as done in the state of the art. The attainment of a precise turbidity value is more reliable starting from low values.

It must be noted that, as a further advantage and setting aside the need to do an anti-biogram, the above mathematical formula also allows to quantify the bacterial count.

This quantification is given by the differential measuring between initial turbidity and final turbidity which is evaluated in combination with other parameters (time, speed of replication of the microorganism). This is useful for the bacterial count which is complex to quantify, for example bacteria with a long growth time, a matter of days, or for those where the duration of bacterial growth depends on the type of bacteria and on the speed of replication, inasmuch as it provides not only positivity, but also a quantitative result.

In other words, the method according to the invention could be used as a prolonged incubation to provide an indication of the entity of the bacterial infection in the culture broth, in other applications or measurements that are not the anti-biogram test.

For example, this is valid for testing bacteria of environmental interest, or in food, or to detect and quantify bacteria in food or animal food matrixes, where the anti-biogram is not provided or useful.

According to a variant embodiment, in said first step a suitable lysing mean is dispensed in the first container in order to obtain lysis of the red corpuscles, with the purpose of freeing and then measuring intracellular bacteria possibly present inside the red corpuscles.

The variant in which we have lysing of red corpuscles allows to test both the extracellular bacteria and also the intracellular bacteria.

The time taken for analysis with the present invention is considerably less than state of the art methods. The speed of detection is possible thanks to a nephelometric type measurement based on light scattering, much quicker and more sensitive in establishing in a short time the presence/absence of bacterial growth in the sample thanks to a direct detection of the turbidity and hence of the concentration of organisms.

The present invention thus allows to provide precise and reliable results with a considerable saving of time compared with known methods, and can also be achieved with pre-existing machines and instruments.

In other words, the method allows to identify all the positives within a period of time that is significantly shorter than in classic methods of haemoculture.

This will allow to significantly reduce the average referral time of the anti-biogram with obvious therapeutic benefits for the patient and the management thereof.

The method according to the invention can be automated, requires limited manual operations, thanks to the automated steps of reading, data processing, display of results, etc.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a block diagram of a method according to the present invention;

FIG. 2 is a schematic representation of the functioning of a method according to the present invention.

### DETAILED DESCRIPTION OF A PREFERENTIAL FORM OF EMBODIMENT

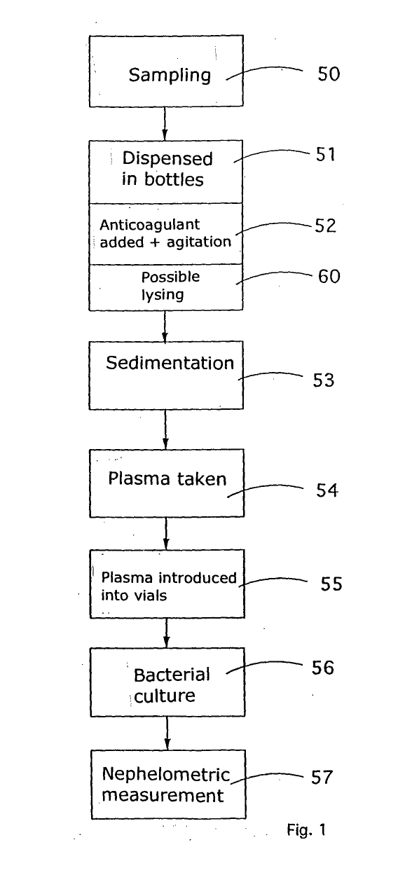

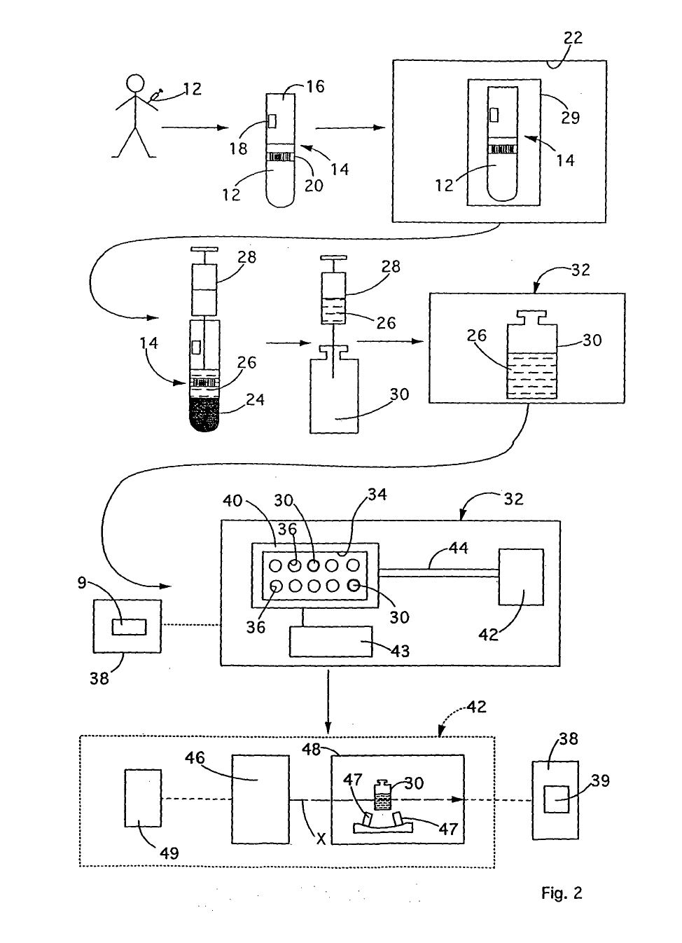

With reference to FIGS. 1 and 2, a method for bacteriological testing on plasma comprises a step 50 of taking a blood sample 12 from the patient and a subsequent dispensation step 51 in which the sample 12 is dispensed in a sterile collection bottle 14 containing an anticoagulant, such as SPS. It is preferable to select a different point for each sampling.

Another necessity is to avoid taking the blood from permanent vein or artery catheters, unless it is impossible to make the intravenous injection or unless there is a suspected sepsis caused by an endovascular catheter.

After having felt the zone of the patient's body where the sample is to be taken, this is carefully disinfected, since for the type of culture used pollution is very easy.

The area is left to dry and the needle is introduced, without touching the disinfected zone again, in order to take the sample.

First of all, the stopper 16 of the bottles 14 into which the blood sample will be introduced is disinfected, and it is left to dry.

There follows a further agitation step 52 in which the bottle 14 is shaken so as to activate the anticoagulant SPS to prevent the formation of coagulation.

A label 18 is stuck on each bottle 14 with the data of the patient carried on a bar code 20. Writings, plasters, labels or other adhesives in the area occupied by the bar code of the bottle are to be avoided. This is to allow the sample 12 to be traced by reading the bar code.

Then the bottle 14 is thermostated at 37° C. in a suitable thermostated container 22.

Optionally, to test intracellular bacteria, a lysis step 60 of the red corpuscles is also provided, using a suitable lysing agent, after the addition of the anticoagulant.

There is a subsequent sedimentation step 53, of the gravitational type, in a suitable sedimentation unit 29 where, after a wait of about 1-3 hours, the plasma is separated, indicated by reference number 26, from the corpuscular part.

According to another embodiment, a centrifuge sedimentation unit can be used.

The test tube is put at an inclination of 45°, to accelerate the separation of the corpuscular part 24 of the blood from the plasma 26.

The method then continues with the sterile sampling step 54, in which about 500-1000 microliters of plasma 26 are taken from the bottle 14 by means of a sterile syringe 28.

Subsequently, there is an inoculation step 55 in which the sample (plasma) is inoculated into vials or test tubes 30 containing a liquid culture broth (eugonic broth) and a subsequent culture step 56. The eugonic broth is suitable for the growth of aerobic microorganisms and for the execution of an optical measurement.

The vials 30 are first sterilized by means of autoclave, and in any case it is recommended to re-sterilize the rubber membrane of the vial 30 before inoculation.

In particular, the vials 30 are used for a light-scattering measurement in a suitable rotor of a nephelometric measuring machine 32.

Advantageously, each vial 30 where the culture and nephelometric measuring are carried out is substantially of homogeneous size and thickness, made of material transparent to electromagnetic radiations for determinate wave lengths, for example such as optical glass or transparent plastic.

According to one embodiment, the bacterial culture takes place inside the nephelometric measuring machine 32 itself.

According to another embodiment, the nephelometric measuring machine 32 comprises a housing 34 with one or more suitable seatings 36 for the vials 30.

A processing unit 38, provided with suitable peripherals, such as video, keyboard, printer etc., cooperates with the housing 34, and automatically starts and manages the whole operating cycle of analysis.

Inside the housing 34 a thermostat device 40 is provided, managed by the processing unit 38, to keep the temperature of the samples constant and controlled, at about 37° C., during the bacterial incubation and growth.

Mechanical, magnetic or other type of agitator means 43 are provided, to allow homogenization and to render uniform the suspension of the bacteria in the plasma inoculated inside each vial 30.

According to a particular solution, each vial 30 is provided inside with a small metal ferromagnetic anchor which is initially resting on the bottom. The metal anchor cooperates with the agitator means 43, in this case provided with magnets which, started by the processing unit 38 at the start of the cycle, draw the metal anchor inside the test tube, allowing homogenization and making the suspension uniform.

The homogenized suspension of the growing bacteria makes the detection independent of the flotations, sedimentations and aggregations that are typical of the way various bacterial species grow.

When several samples (vials) are loaded, the nephelometric measuring machine 32 comprises a mobile unit 44 that allows to read the individual sample at pre-established intervals and for pre-established times by means of a light-scattering nephelometric reading device 42 of the nephelometric measuring machine 32.

The reading device 42 is provided with a focusing and collimation device 46 and a detection device 48.

The focusing and collimation device 46 is associated with a device 49 to generate electromagnetic radiation, generated according to an emission axis X.

The electromagnetic radiation generator 49 sends the radiation to the focusing and collimation device 46 which the reading device 42 aligns with respect to the vial 30.

According to the present invention, the electromagnetic radiation can be polarized or not.

The radiation emitted by the focusing and collimation device 46 is diverted by the sample and collected by the detection device 48.

Throughout the incubation in the culture step 56, the detection device 48 detects any possible bacterial growth, by means of the nephelometric reading of the sample, and shows the curves of bacterial growth detected (calculation step 57) and calculates the final bacterial load by means of the nephelometric measuring machine 32. The samples showing positive to having reached McFarland turbidity 0.5 are signaled, either on the monitor or acoustically, in order to perform the suitable tests for the clinical anti-biogram.

In fact, once a sample has been detected as positive to culture, it is possible, by determining the McFarland 0.5 on the eugonic culture broth, to effect the anti-biogram directly, using the same eugonic broth at 0.5 McFarland as inoculum.

For this technique it will not therefore be necessary to have the classic isolation step, obtained by sowing the broth on Petri dishes and subsequently preparing the inoculum by means of dilution, in a physiological/saline solution of the isolated colonies, until 0.5 McFarland turbidity.

When the detection of the sample is terminated, the mobile unit 44 takes the reading device 42 into cooperation with the next vial 30.

According to a first solution, the detection device 48 comprises a detector 47 that at least during the examination period relating to the individual test tube is situated at a fixed angle with respect to the optical axis.

According to a variant, the detection device 48 comprises a plurality of detectors 47 (two are shown in FIG. 2), situated a different angles with respect to the optical axis of the collimation and focusing system.

When there are several detectors 47, they can be of the specific type positioned in a defined angle, or the continuous type, able to cover the whole angle practically without a break in continuity.

Advantageously, the detectors 47 are disposed so as to cover substantially an angle variable between 0° and 180° with respect to the optical axis, since due to considerations of symmetry, the information obtained with the detectors located so as to cover said angles provides all the information needed for the analysis of the sample in question.

The radiation is read in succession at desired intervals by the detection device 48 according to a defined angle, or according to defined angles comprised between 0° and 180° with respect to the axis of emission, according to the two alternatives, with a consequent construction of the curve that represents the intensity of the radiation diverted, with respect to time, correlated to the bacterial growth.

The radiations detected by the detectors are converted into electric signals and then sent to the processing unit 38 which processes the data and calculates the results.

In particular, the curve of bacterial growth is compared with reference values comprised in a data bank 39 of the processing unit 38 in order to determine the typical analysis parameters, such as quantity, speed of replication and morphology of the microorganisms present in the sample.

The calculation procedure is based on the fact that the bacterial growth inside the suspension causes variation over time of the intensity of diverted light.

Periodic readings of the diverted radiation allow to construct, by means of known interpolation procedures, the growth curve of the bacterial colonies inside the blood sample examined, said curve relating to the angle of detection in which the detector is positioned.

It has been shown experimentally that the growth curve has an exponential development as a function of time, of the type C_{B}=Ae^{K}^{ₙ}^{(t-t}^{₀}⁾+C.

In the formula, C_{B} represents the intensity of the radiation diverted, A and C are constants depending respectively on the bacterial species examined and on the initial concentration, Kₙ is a parameter which takes into account the angle of positioning of the detector, t is the time and t₀ is a delay connected to the number of bacteria present in the sample

By correlating the characteristic parameters of the formula C_{B}=Ae^{K}^{ₙ}^{(t-t}^{₀}⁾+C, such as A, C, Kₙ, with standard values obtained experimentally and memorized in a data bank in the processing unit, it is possible to obtain information useful for identifying the bacterial species present inside the solution.

The data bank 39 is constructed using for example one of the following two procedures.

The first procedure provides to acquire samples of bacterial species already identified (for example ATCC strains), to insert them into the apparatus according to the invention and to construct the characteristic curve relating to that particular bacterial species.

The second procedure uses a sample with a bacterial species to be identified and, after separating it, for example into two halves, the first half is analyzed using the traditional method, for example Petri dishes, and the second half with the method according to the invention.

In this way it is possible to associate with every bacterial species identified by the traditional method a particular growth curve obtained with the method according to the invention.

The data bank is constructed, by means of the same method, keeping in mind the type of bacteria, using a large number of said procedures, for example in the order of hundreds for each characteristic detection angle.

In this way, for each bacterial species, it is possible to evaluate the parameters of the formula C_{B}=Ae^{K}^{ₙ}^{(t-t}^{₀}⁾+C, for example A, C, Kₙ, etc.

In the course of analysis of the sample examined, the processing unit 38 calculates the parameters (formula C_{B}=Ae^{K}^{ₙ}^{(t-t}^{₀}⁾+C) of the growth curve obtained and compares them with the parameters memorized in the data bank 39 and relating to the various bacterial species.

From this comparison the processing unit 38 provides, with good reliability, the identification of the bacterial species present in the sample of plasma.

Given that the characteristic of the growth curve is closely dependent on the angle of detection, the monitoring angles must be the same as those used to create the data bank.

Compared with using a single detector positioned at a determinate angle, the application of several detectors at different angles allows to obtain more information concerning the anisotropy of the signal, which is closely correlated to the morphology of the microorganisms present in the sample of plasma.

With reference to the formula C_{B}=Ae^{K}^{ₙ}^{(t}^{₀}⁾+C, the parameters C_{B} and t₀ depend on the number of bacteria present in the sample.

The number of bacteria, by means of t₀, conditions the evolution of the growth curve, which is obtained by processing the intensity of the diverted radiation.

The whole calculation procedure is automated and the processing unit 38, having started the cycle, will provide at output, after the necessary time and by means of video or printer, all the desired information, such as initial bacterial concentration, speed of growth, information on the type of bacteria, etc.

Other forms of embodiment of the present invention provide to incubate the blood sample, for example 5-10 ml, in a bottle for haemoculture. As soon as the sample proves positive to growth, the bottle with the positive haemoculture is selected and a quantity of the content is taken, for example 2 ml of broth, to subject it to sedimentation by centrifugation. Afterward, a solution is prepared with a suitable 0.5 McFarland turbidity, for example by inoculating about 100 microliters of plasma in a vial of eugonic broth to obtain said suitable turbidity, using a turbidometer. Then the direct anti-biogram is prepared, without waiting for the identification of the bacterium, using a personalized antibiotic panel (comparison with reference sample and test tubes with antibiotics). Finally, the sample is processed, obtaining results in three hours in all, instead of the two days as in the classic Kirby Bauer method.

It is clear that modifications and/or additions of parts may be made to the method for bacteriological testing on plasma as described heretofore, without departing from the field and scope of the present invention.

It is also clear that, although the present invention has been described with reference to specific examples, a person of skill in the art shall certainly be able to achieve many other equivalent forms of method for bacteriological testing on plasma, having the characteristics as set forth in the claims and hence all coming within the field of protection defined thereby.

1. A method for bacteriological testing on plasma, comprising the following steps:
a first step in which a blood sample taken from a patient is dispensed in a first container;
a second step in which the sedimentation of the blood sample is determined, so as to separate the corpuscular part, which sediments on the bottom of the first container from the liquid part or plasma which represents the surnatant;
a third step in which a determinate portion of the surnatant is taken, consisting of the liquid part or plasma thus obtained;
a fourth step in which the portion of the liquid part or plasma obtained in a culture ground is inoculated inside a second container suitable to allow a bacterial culture and an instrument reading by means of an optical measurement machine;
a fifth step in which bacterial growth is allowed in the culture ground contained in the second container;
a sixth step in which, by means of the optical measurement machine, on the culture ground contained in the second container, an optical measurement is made in order to detect and/or quantify the presence of bacteria and microorganisms.

2. The method as in claim 1, wherein the optical measurement of the sixth step takes place simultaneously with the fifth step, so as to measure the bacterial growth directly.

3. The method as in claim 1, wherein the optical measurement of the sixth step is able to signal that the culture ground has reached 0.5 turbidity level on the McFarland scale, so that the anti-biogram can be carried out directly, using the same culture ground as the inoculum.

4. The method as in claim 1, wherein the detection and/or identification is targeted at least on an extra-cellular search of red corpuscles, bacteria and aerobic microorganisms, microaerophiles or capnophiles, present in the liquid part or plasma.

5. The method as in claim 1, wherein the culture ground is of the liquid type.

6. The method as in claim 1, wherein the optical measurement that is carried out is of the nephelometric type based on the light scattering technique.

7. The method as in claim 1, wherein the optical measurement carried out by the optical measurement machine is of the kinetic type with fixed timing, based on the light scattering technique, in order to determine the presence of possible bacterial growths, and subsequently, by analyzing the signals, to reveal the bacterial growth curves.

8. The method as in claim 1, wherein the second container is of the test tube type made of material transparent to defined electromagnetic wave radiations,

9. The method as in claim 1, wherein the second container with the inoculated culture ground and housed in the optical measurement machine cooperates with a thermostat device and at least temporarily with an agitator unit to be subjected to thermostating and continuous mixing, in order to promote the possible growths of the microorganisms present in the liquid part or plasma.

10. The method as in claim 1, wherein said second container also cooperates with a focusing and collimation device and a detection device, the focusing and collimation device being able to emit a defined electromagnetic wave radiation, with its own axis of emission (X), which is transmitted through the sample, wherein the electromagnetic wave radiation is diverted by the bacteria present in the sample with an intensity that depends on their number and morphology, the diverted radiation being subsequently detected with desired cadences by the detection device with a consequent construction of a bacterial growth curve, said growth curve being compared by a processing unit with reference values comprised in a data bank of the processing unit so as to quantify the bacterial load and to identify the bacterial species according to the comparison with growth curves obtained from the data bank.

11. The method as in claim 10, wherein the curve that represents the growth of the bacteria as a function of time is expressed in analytical form according to the formula C_{B}=Ae^{K}^{ₙ}^{(t-t}^{₀}⁾+C. where C_{B} represents the intensity of the radiation diverted, A and C are constants depending respectively on the bacterial species examined and on the initial concentration, Kₙ is a parameter which takes into account the angle of positioning of the detector, t is the time and t₀ is a delay connected to the number of bacteria present in the sample.

12. The method as in claim 1, wherein the first step, provides to add an anticoagulant to the first container, and subsequently to agitate the anticoagulant to prevent the coagulation of the sample.

13. The method as in claim 1, wherein in the first step a lysis is carried out of the red corpuscles of the sample, with the purpose of freeing and then measuring bacteria possibly present inside the red corpuscles, by means of a lysing means provided or introduced into the first container.

14. A method for bacteriological testing on plasma, comprising the following steps:
a first step in which a blood sample taken from a patient is dispensed in a first container, containing a lysing means, in order to obtain a lysis of the red corpuscles, with the purpose of freeing and then measuring bacteria possibly present inside the red corpuscles;
a second step in which the sedimentation of the lysed erythrocytes present in the blood sample is determined, so as to separate the corpuscular part, which sediments on the bottom of the first container, from the liquid part or plasma which represents the surnatant;
a third step in which a determinate portion of the surnatant is taken, consisting of the liquid part or plasma thus obtained;
a fourth step in which the portion of the liquid part or plasma obtained in a liquid culture ground is inoculated inside a second container suitable to allow a bacterial culture and an instrument reading by means of an optical measurement machine;
a fifth step in which bacterial growth is allowed in the culture ground contained in the second container;
a sixth step in which, by means of the optical measurement machine, on the culture ground contained in the second container, an optical measurement is made in order to determine the presence of bacteria and microorganisms.

15. The method as in claim 13, wherein the analysis is targeted on an extra-cellular and intra-cellular search of red corpuscles, bacteria and aerobic microorganisms, microaerophiles or capnophiles, present in the liquid part or plasma.

16. An apparatus for making a bacteriological test on plasma, comprises comprising a sedimentation unit for a blood sample, taken from a patient and contained in a first container, so as to separate the corpuscular part of the blood sample, which sediments on the bottom of the first container, from the liquid part or plasma which represents the surnatant, pick-up and inoculum means to pick up a determinate portion of the surnatant, consisting of the liquid part or plasma thus obtained, and to inoculate the portion of the liquid part or plasma obtained in a culture ground in a liquid state, inside a second container suitable to allow a bacterial culture and an instrument reading of an optical type, the culture ground being able to allow bacterial growth in the second container, optical measurement means, to effect an optical measurement of the culture ground contained in the second container, in order to determine the presence of bacteria and microorganisms, and processing means comprising a data bank, to collect the data of the optical measurement, to construct a curve that represents the intensity of the radiation diverted by the culture ground in the optical measurement with respect to time, whose parameters are compared with reference values contained in the data bank, in order to determine typical analysis parameters, said values being characteristic for each bacterial species.

17. The apparatus as in claim 16, wherein the second container is of the test tube type made of material transparent to defined electromagnetic wave radiations.

18. The apparatus as in claim 16, comprising a thermostat device and an agitator device able to cooperate with the second container.

19. The apparatus as in claim 16, comprising a focusing and collimation device and a detection device able to cooperate with the second container, the focusing and collimation device being able to emit a defined electromagnetic wave radiation, with its own axis of emission (X), which is transmitted through the sample, wherein the electromagnetic wave radiation is diverted by the bacteria present in the sample with an intensity that depends on their number and morphology, the diverted radiation being subsequently detected with desired cadences by the detection device (48) with a consequent construction of a bacterial growth curve, and in that it also comprises a processing unit with a data bank having reference values by means of which to compare said growth curve so as to quantify the bacterial load and to identify the bacterial species according to the comparison with growth curves obtained from the data bank.
